(19) United States
(12) Patent Application Publication
(10) Pub. No.: US 2004/0115163 A1
(43) Pub. Date: Jun. 17, 2004

(54) Cosmetic product

(76) Inventors: Antoine Gedouin, Saint-Malo (FR); Romuald Vallee, Saint-Meloir-des-Ondes (FR); Pierre-Yves Morvan, Rennes (FR)

Correspondence Address: LOWE HAUPTMAN GILMAN AND BERNER, LLP, 1700 DIAGONAL ROAD, SUITE 300 /310, ALEXANDRIA, VA, 22314, US

(21) Appl. No.: 10/466,855

(22) PCT Filed: Jan. 22, 2002
(86) PCT No.: PCT/FR02/00286

(30) Foreign Application Priority Data
Jan. 22, 2001 (FR) 01 00905

(51) Int. Cl. A61K 35/78; A61K 7/06

(52) U.S. Cl. 424/74; 424/725; 424/764

(57) ABSTRACT

A cosmetic product to retard skin aging and comprising vegetal lipidic extracts belonging to the families of the apiaceae, cistaceae, asteracea or lamiaceae.

The present invention relates to a cosmetic or pharmaceutical product containing plant lipid extracts controlling the division and differentiation of human epidermic cells. Moreover the invention relates to a nutritional supplement containing such extracts.

The epidermis is a dynamic structure of the human skin. Essentially it consists of a single cell family, namely the keratinocytes. These are produced by a layer of stock cells at the base of the epidermis and they differentiate with respect to thickness across the epidermal thickness: initially they are nearly cubic, then they become polyhedral and then flatten and terminate at the surface by producing a fairly thick calloused layer. Accordingly the epidermis produces cell multiplication at the base layer and a cell differentiation zone at the middle zone resulting in a well differentiated protective zone (callous layer).

Stock cell division is regulated by cytokines and growth factors such as the Epidermal Growth Factor EGF. It is known that, contrary to the case of steroid hormones and lipid origin vitamins which are able to cross the plasma membrane to affix themselves on nuclear receptors, the cytkines and growth factors will act through membrane receptors.

Among the therapeutic substances that might regulate proliferation and differentiation of keratinocytes, retinoic acid (RA) and its derivatives (retinoids) have been used for several years already. However they incur the drawback that they are teratogenic and exhibit cutaneous intolerance. Applying retinoic acid to the skin decreases the expression of proteins which typically will be expressed during the differentiation of epidermal cells, in particular cytokeratin 1 (CK1), transglutamine 1 (TGM1) and desmoplakins (DP). Retinoic acid acts through nuclear receptors (RAR, RXR), furthermore also through cytoplasmic proteins. Indeed proteins termed CRABP-I and CRABP-II which specifically bind cellular retinoic acid, namely cellular retinoic acid binding proteins I and II) have been detected in the cytoplasm of cells sensitive to retinoic acid. The CRABP-II protein is the product of a gene which is mainly expressed during embryonic genesis, specifically in the growth of the nervous system and of the face, but which continuous being expressed in the adult in particular in the skin cells. The expression of the NRA messengers of CRABP-II is specifically induced by retinoic acid and is regulated by cytokines such as interleukin-1 that are known being significant in epidermal differentiation. The CRABP-II proteins regulate intercellular retinoic acid concentration and moreover its transport and its metabolism: the CRAB-II protein modifies the expression of retinoic-acid sensitive genes regulating proliferation and differentiation.

The basic objective of the present invention is a substance regulating keratinocyte proliferation and differentiation while free from the undesirable effects encountered with retinoic acid.

This problem was solved by discovering properties similar to those of retinoic acid and the growth factor EGF in lipidic extracts of certain so-called sea shore plant families such as Crithmum maritimum (sea fennel), Cistus monspeliensis, Helichrysum italicum and Lavandula stoechas.

In surprising manner, it could be shown that some of these plant extracts, even though lacking pro-vitamin A (a vegetal retinoid), might exhibit a regulatory action similar to that of retinoic acid without however entailing inflammation.

An extract was processed for each the plants Crithmum maritum, Cistus monspeliensis, Helichrysum italicum and Lavandula stoechas by super-critical CO₂ co-extraction in the presence of a solvent composed of triglycerides of vegetal origin (C8-C10 TG). Supercritical CO₂ and the vegetal co-solvent offer the advantage of stabilizing the extract without degrading it. However other kinds of solvents may also be considered for extracting.

After returning to room temperature and evaporating the CO₂, the extract is solidified in the form of a wax by adding a vegetal oil (C16-C18 TG) which is solid at room temperature. The wax so made offers the advantage of being oxidation resistant.

In the manner shown farther below, it was found that these waxes favor expressing coding genes as regards those proteins that are typically stimulated by retinoic acid and its derivatives.

Accordingly processing by means of waxes of sea fennel, of Cistus monspeliensis and of Helichrysum italicum increases the expression of the coding CRABP-II gene as regards the cell protein binding retinoic aid (cellular retinoic acid-binding protein II or CRABP-II) and enhances cell differentiation. Moreover, and just as for the retinoic acid and its derivatives, the waxes so made enhance expression of genes which code resp. for the vascular endothelial growth factor (VEGF) or its receptor (VEGFR1), making possible positive regulation of the division of basal cells.

At the same time, it could be shown that the waxes so made inhibit the expression of protein coding genes usually expressed during epidermal differentiation. Such repression also is a feature of retinoic acid and its derivatives.

Illustratively, treatment by the waxes of sea fennel, of Cistus monspeliensis, Helichrysum italicum and Lavandula stoechas will decrease the expression of genes coding for intracellular cohesion proteins such as desmoplakin 1, cytokeratin 1, cytokeratin 6 or enzymatic proteins such as transglutaminase 1.

The waxes of sea fennel, of Cistus monspeliensis, Helichrysum italicum and Lavandula stoechas therefore exhibit effects which are similar to those of the retinoids as regards positive or negative regulation of keratinocyte differentiation.

On the other hand it was shown in significant manner that, contrary to the case of retinoic acid, the waxes will not induce inflammation reactions because the genes that code the inflammatory cytokines, namely interleukin-1 alpha (IL-1A) and beta (IL-1B) will not be stimulated. On the contrary, a tendency toward increasing the expression of the gene coding the receptor antagonist to IL-1 alpha (IL-1RA) was in fact observed for the waxes of sea fennel, Cistus monspeliensis and Lavandula stoechas, indicating anti-inflammation action.

Furthermore it was discovered that the waxes made from sea fennel, Cistus monspeliensis and Lavandula stoechas increase the expression of the coding gene for an EGF response factor (EGF response factor 1 or ERF1).

Moreover it was noted that the wax made from Cistus monspeliensis enhances the expression of genes coding for the epidermal growth factor receptor or EGFR.

Lastly it was noted that the waxes of sea fennel, Helichrysum italicum and Lavandula stoechas increase substantially the expression of the gene coding for a growth factor of the EGF type binding heparin (heparin binding EGF-like growth factor or HB-EGF). That gene moreover is also stimulated by retinoic acid.

In conclusion, therefore, the above vegetal waxes act both on epidermal differentiation (inhibition and stimulation being similar to those of the retinoids) and on keratinocyte division (stimulation similar to the EGF factor).

By stimulating the stock cells' division and by regulating the differentiation of the keratinocytary cells, the vegetal waxes enhance thickening and maturation of the epidermis. They may be used as the active principle in cosmetic products, in particular to fight the aging effects of the skin cells. They also may be used in dermatology as the active principle of a pharmaceutical product to treat the disorders of keratinocyte differentiation, in particular as an anti-psoriatic agent. Prophylactic treatment also may be considered. Furthermore, thanks to their stimulating cell division in a manner similar to that of EGF, the vegetal waxes moreover may be used as the active principle of a pharmaceutical used in speeding up scar formation.

The vegetal waxes also may be incorporated into foodstuffs in order to attain the above cosmetic effect.

In general the vegetal waxes are made from the following plants:

Apiaceae family, in particular the Crithmum genus, for instance from Crithmum maritium,

Cistaceae family, in particular the Cistus genus, for instance Cistus monspeliensis,

Asteraceae family, in particular the Helichrysum genus, illustratively from Helichrysum italicum,

Lamiaceae family, in particular the Lavandula genus, for instance from Lavandula stoechas.

Experimental Procedure

The waxes made from Crithmum maritium, Cistus monspeliensis, Helichrysum italicum and Lavandula stoechas were diluted to 1% in the solvent used for extraction and then were applied to surfaces of reconstituted human epidermises (in duplicate) and were spread apart using thin, sterile brushes. The epidermises are incubated for 24 h at 37° C. Next the tissues were rid of their carene and immediately placed in sterile tubes (RNAse free) and frozen at −80° C.

The effect of the above waxes on gene expression was investigated on the basis of an analysis of the messenger ribonucleic acid (NRAm).

First the ribonucleic acids were extracted and purified conventionally and then they were analyzed by specific hybridization with probes affixed to a membrane (so-called micro-array method).

More specifically, first the DNA was eliminated from the samples by incubation in the presence of Dnase 1. Absence of residual DNA was verified by electrophoresis on agarose gel.

The NRA messengers (NRAm) then were purified by hybridizing the NRAm poly(A) tails with biotinylated oligo(dT) primers followed by a stage of selective capture on streptavidin balls.

Multiple DNA probes marked with ³²P were prepared by reverse transcribing the NRAm bound to the poly(dT) balls using a pool of NRA primers which were specific to the sequences immobilized on the arrays in the presence of [a ³²P]-dATP.

cDNA's immobilized on each membrane were hybridized overnight at 68° C. with the corresponding, marked probes. The filters then were washed under rigorous conditions (68° C.) and placed in individual plastic pouches for purposes of analysis. Analysis was implemented by directly quantifying the spots' radioactivity.

Results

The results are listed in Table I. The various columns are denoted as follows:

C is a control epidermis processed with the extraction solvent (C8-C1O TG) used to dilute the vegetal waxes, Sea Fennel is an epidermis treated with sea fennel wax diluted to 1 % in the extraction solvent (C8-C10 TG),

Cistus is an epidermis treated with the Cistus monspeliensis wax diluted to 1 % in the extraction solvent (C8-C10 TG),

Heli is an epidermis treated with the Helichrysum italicum wax diluted to 1% in the extraction solvent (C8-C10 TG),

Lav is an epidermis treated with the Lavandula stoechas wax diluted to 1% in the extraction solvent (C8-C10 TG),

RA is the effect of retinoic acid on gene expression as known in the state of the art.

The different rows relate to different markers or, equivalently, to the genes corresponding to these markers.

The results are stated (figures on left) in relative units RU. They state the mean radioactivity of the double spot points corresponding to each gene and corrected for the background noise and the probes' different marking intensities. They are also stated (figures on the right) as percentages of the untreated control.

It is assumed that a gene is sufficiently expressed when its RU is equal to or larger than a threshold of 1.5, that is when the doublet is visible in the pictures.

The reference markers (housekeeping) are ubiquitin and glyceraldehyde-3-phosphate dehydrogenase (G3PDH). As shown in the table's first two rows, these genes were clearly displayed visually and it is easily shown that no wax significantly affected the reference genes' expressions. The mean count of the housekeeping markers was taken as the reference quantifying in relative manner the expressions of the other markers. In this manner the relative variations in marking intensities of the different probes are without weight. Correction was based on the marking intensities of the reference genes of the different probes.

A product is considered repressive if the measured level is at least 50% less than that of the control (i.e., the ratio being less than 50%) and the product is considered stimulative if said level exceeds by more than 50% that of the control (i.e. if its ratio is larger than 150%).

Regarding the cellular protein binding retinoic acid (CRABP-II), an increase in expression of the CRABP-II gene of +113%, +86% and +183% relative to the control is observed (resp. ratios of 213%, 186% and 283) for the following waxes: sea fennel, Cistus monspeliensis and Helichrysum italicum. The Lavandula stoechas wax also tends to increase this gene (+43%).

The sea fennel, Cistus monspeliensis, Helichrysum italicum and Lavandula stoechas waxes lower the expression of the coding genes for expressed proteins as regards epidermal differentiation: desmoplakin 1 (−80% and −74% for the Helichrysum italicum and the Lavandula stoechas waxes), desmoplakin 3 (−60% and −59% for the Cistus monspeliensis and Helichrysum italicum waxes), cytokeratin 1 (−90% for the 4 vegetal waxes), cytokeratin 6 (−79% for the Cistus monspeliensis wax), transglutaminase 1 (−65% and - 61% for the sea fennel and Helichrysum italicum waxes).

Lastly the four vegetal waxes increase the expression of the genes coding for the growth factors: VEGF (+99% and +58% for sea fennel and Cistus monspeliensis) and VEGFR1 (from +55% to +186%).

On the other hand the waxes do not stimulate the production of inflammatory cytokines, namely interleukin-1 alpha (IL-1A) or beta (IL-1B). On the contrary, the waxes of sea fennel, Cistus monspeliensis and Lavandula stoechas tend to stimulate the expression of the gene coding the antagonist of the receptor at IL-1 alpha (1L-1RA) (resp. +45%, +29%, +40%).

Regarding the epidermal growth factors, the waxes of sea fennel, Cistus monspeliensis and Lavandula stoechas increase resp. by +74%, +195% and +53% the coding gene expression for a response factor at EGF (ERF1), the Cistus monspeliensis wax increasing by +57% the coding gene expression for the EGFR and the waxes of sea fennel, Helichrysum italicum and Lavandula stoechas resp. increasing by +120%, +140% and +194% the expression of the gene coding the growth factor HB-EGF.

TABLE 1

| | | SEA | | | | | | | | |
|---|---|---|---|---|---|---|---|---|---|---|
| | C | FENNEL | | CISTUS | | HELI | | LAV | | |
| MARKER | RU | RU | % | RU | % | RU | % | RU | % | RA |
| ubiquitin | 551.1 | 671.5 | 122 | 652.2 | 118 | 694.4 | 126 | 845.1 | 117 | |
| glyceraldehyde 3-phosphate | 29.7 | 36.4 | 122 | 35.9 | 121 | 31.8 | 107 | 46.3 | 156 | |
| dehydrogenase (G3PDH) | | | | | | | | | | |
| cellular retinoic acid-binding | 3.5 | 7.4 | 213 | 5.5 | 186 | 9.9 | 283 | 5.0 | 143 | + |
| protein II (CRABP-II) | | | | | | | | | | |
| desmoplakine I & II (DPI & | 33.7 | 20.1 | 60 | 38.1 | 113 | 5.7 | 20 | 8.8 | 26 | − |
| DPII) | | | | | | | | | | |
| desmoplakine III (DP3) | 68.4 | 47.1 | 69 | 21.4 | 31 | 28.1 | 41 | 41.0 | 60 | − |
| transglutaminase 1 (TGM1) | 30.6 | 10.8 | 35 | 15.0 | 49 | 12.0 | 39 | 17.1 | 56 | − |
| cytokeratine 1 (CK1) | 389.9 | 23.2 | 6 | 25.0 | 6 | 12.3 | 3 | 35.7 | 9 | − |
| cytokeratine 6 (CK6) | 127.1 | 78.2 | 62 | 27.2 | 21 | 115.7 | 91 | 119.2 | 94 | − |
| Interleukine-1 alpha (IL-1A) | 3.4 | 3.0 | 88 | 2.8 | 83 | 2.8 | 83 | 2.6 | 78 | + |
| Interleukine-1 beta (IL-1B) | 2.4 | 3.1 | 126 | 2.3 | 96 | 2.2 | 91 | 2.2 | 89 | |
| Interleukine-1 receptor | 9.1 | 13.3 | 145 | 11.8 | 129 | 7.4 | 81 | 12.8 | 140 | |
| antagonist protein (IL-1RA) | | | | | | | | | | |
| epidermal growth factor | 1.0 | 1.4 | 132 | 1.6 | 157 | 1.1 | 106 | 1.3 | 125 | |
| receptor (EGFR) | | | | | | | | | | |
| EGF response factor 1 | 9.8 | 17.1 | 174 | 28.0 | 295 | 9.9 | 100 | 15.0 | 153 | |
| (ERF1) | | | | | | | | | | |
| heparin-binding EGF-like | 5.7 | 12.5 | 220 | nd | nd | 13.7 | 240 | 16.7 | 294 | + |
| growth factor (HBEGF) | | | | | | | | | | |
| vascular endothelial growth | 6.0 | 11.9 | 199 | 9.4 | 158 | 3.1 | 52 | 7.6 | 127 | |
| factor precursor (VEGF) | | | | | | | | | | |
| vascular endothelial growth | 3.1 | 6.8 | 221 | 8.8 | 286 | 5.9 | 191 | 4.8 | 155 | |
| factor receptor 1 (VEGFR1) | | | | | | | | | | |

1. A cosmetic product to retard skin aging, characterized in that
it comprises lipidic extracts prepared from one or more of the Crithmum maritimum, Cistus monspeliensis and Helichrysum italicum plants.

2. Cosmetic product as claimed in either of claims 1 and 2, characterized in that it is in the form of a vegetal wax.

3. Manufacturing procedure of a cosmetic product retarding skin aging, characterized in that
lipidic compounds are extracted from one or more of the Crithmum maritimum, Cistus monspeliensis and Helichrysum italicum plants, where said extraction is by means of supercritical CO₂ co-extraction in the presence of a solvent composed of vegetal origin triglycerides.

4. Manufacturing procedure for a cosmetic product as claimed in claim 3, characterized in that a vegetal oil solid at room temperature is added to said lipidic compounds.

5. A pharmaceutical compound to dermatologicallytreat skin aging, characterized in that
it comprises lipidic extracts prepared from one or several of the Crithmum maritimum, Cistus monspeliensis and Helichrysum italicum plants.

6. Pharmaceutical product as claimed in either of claims 1 and 2, characterized in that it is in the form of a vegetal wax.

7. A manufacturing procedure for a pharmaceutical product to treat skin aging, characterized in that
the lipidic compounds are extracted from one or several Crithmum maritimum, Cistus monspeliensis and Helichrysum italicum plants by supercritical CO₂ co-extraction in the presence of a solvent composed of vegetal origin triglycerides.

8. Manufacturing procedure of a pharmaceutical product as claimed in claim 7, characterized in that a vegetal origin oil solid at room temperature is added to said lipidic compounds.

9. A cosmetically acting food supplement, characterized in that
it comprises lipidic extracts prepared from one or several of the Crithmum maritimum, Cistus monspeliensis and Helichrysum italicum plants.

10. Food supplement as claimed in claim 9, characterized in that it is in the form of a vegetal wax.

11. A manufacturing procedure of a dermatologically active food supplement, characterized in that
the lipidic compounds are extracted from one or several of the Crithmum maritimum, Cistus monspeliensis and Helichrysum italicum by supercritical CO₂ co-extraction in the presence of a solvent composed of vegetal origin triglycerides.

12. Manufacturing procedure as claimed in claim 11, characterized in that a vegetal origin oil solid at room temperature is added to said lipidic compounds.
